(19) United States
(12) Patent Application Publication
(10) Pub. No.: US 2025/0268736 A1
(43) Pub. Date: Aug. 28, 2025

(54) VASCULAR STENT AND MANUFACTURING PROCESS THEREFOR

(71) Applicant: THE SECOND AFFILIATED HOSPITAL OF GUANGZHOU MEDICAL UNIVERSITY, Guangzhou (CN)

(72) Inventors: Shaojun LIU, Guangzhou (CN); Shiming LIU, Guangzhou (CN); Jing CHEN, Guangzhou (CN)

(21) Appl. No.: 18/741,802

(22) Filed: Jun. 13, 2024

(30) Foreign Application Priority Data
Feb. 22, 2024 (CN) 202410199022.9

(51) Int. Cl. A61F 2/915; A61K 31/436; A61K 45/06

(52) U.S. Cl. CPC A61F 2/915; A61K 31/436; A61K 45/06

(57) ABSTRACT

The present disclosure provides a vascular stent and a manufacturing process therefor, which belong to the technical field of medical apparatuses and instruments. The vascular stent provided by the present disclosure is loaded with an endothelial protein C receptor (EPCR) activator (including activated protein C, TR47 and parmodulin 2) and a limus drug, and the manner of loading includes but not limited to spraying and wrapping.

### TECHNICAL FIELD

The present disclosure relates to the technical field of medical apparatuses and instruments, and in particular to a vascular stent and a manufacturing process therefor.

### BACKGROUND

Cardiovascular and cerebrovascular accidents are the most common cause of death worldwide. Vascular interventional therapy is a common method to restore arterial blood flow, including arterial balloon angioplasty, vascular stent implantation, etc. Drug-eluting stents (DES) are widely used in cardiovascular and cerebrovascular events. There are two main commercial types of DES, namely paclitaxel and limus. Currently, the limus, including rapamycin (sirolimus), as well as various derivatives of rapamycin, everolimus and zotarolimus, is dominant. Limus inhibits the activation of mTOR in vascular smooth muscle cells (SMC), prevents the progression of the cell cycle from G1 to S, and prevents SMC multiplication, thereby preventing vascular restenosis. Paclitaxel alters the interaction between β-tubulin and microtubules, induces a cell cycle arrest, and inhibits SMC multiplication, migration, intimal hyperplasia, and vascular restenosis.

The use of the DES significantly reduces the incidence of in-stent restenosis compared with early bare-metal stents (BMS), but significantly increases the risk^{1,2} of stent thrombosis (ST) in late (1 month to 1 year) and very late (>1 year) periods. ST is one of the most serious complications after vascular intervention, with high mortality. ST is primarily due to non-selective inhibition of endothelial cells (ECs) by stent drugs (limus or paclitaxel), which delays the re-endothelialization process of the stent. The part of the stent not covered by EC in the vessel is continuously exposed and acts as a foreign body to repeatedly irritate the body, eventually forming thrombus. In addition, rapamycin, everolimus, zotarolimus, and paclitaxel increase tissue factor activity and promote thrombosis³. The traditional DES focus only on the effect of inhibiting restenosis and neglects the effect of preventing ST.

In recent years, bioresorbable stents (BRS) have been found to be more conducive to active vascular remodeling. However, despite good results have been realized in other areas, there is considerable debate⁴⁻⁸ in many clinical studies as to whether BRS reduce the incidence of ST and subsequent sudden cardiac death, etc. Therefore, there is an urgent need to develop a novel vascular stent having dual effects that can inhibit SMC (thereby preventing restenosis), promote functions of ECs, and promote a re-endothelialization process of the stent (thereby preventing ST).

### SUMMARY

It is found that limus and paclitaxel can inhibit expression of an endothelial protein C receptor (EPCR) in a vascular endothelial cell membranes.

In view of this, an objective of the present disclosure is to provide a vascular stent and a manufacturing process therefor. The vascular stent provided by the present disclosure is loaded with an endothelial protein C receptor (EPCR) activator and a limus drug, which can not only inhibit vascular restenosis but also prevent stent thrombosis.

To achieve the above objective, the present disclosure provides the following technical solutions:

A vascular stent is provided. The vascular stent is loaded with an endothelial protein C receptor (EPCR) activator and a limus drug, and the manner of loading includes but not limited to spraying and wrapping.

Optionally, the EPCR activator is one or more of activated protein C, TR47 and Parmodulin 2.

Optionally, the limus drug includes one or more of rapamycin, everolimus, zotarolimus, tacrolimus, deforolimus, biolimus A9 and pimecrolimus.

Optionally, the spraying includes but not limited to spraying a coating of an EPCR activator and a limus drug on a metal stent, and the wrapping includes but not limited to wrapping the activated protein C and the limus drug in a bioresorbable stent.

Optionally, spraying amounts of the EPCR activator and the limus drug are 1 μg/mm-100 μg/mm and 1 μg/mm-100 μg/mm respectively.

Optionally, the wrapping amounts of the EPCR activator and the limus drug are 1 μg/mm-100 μg/mm and 1 μg/mm-100 μg/mm respectively.

The present disclosure further provides a manufacturing process for the vascular stent mentioned in the above technical solution. The manufacturing process includes the following steps:

S1, performing stent pretreatment:

S2, preparing an EPCR activator-limus drug solution: dissolving the limus drug and the EPCR activator in a tetrahydrofuran solution containing 10% of poly(lactic-co-glycolic acid) (PLGA); and

S3, performing rotary spraying.

Optionally, final concentrations of the limus drug and the EPCR activator in S2 are both 50 μg/mL.

Beneficial effects: the vascular stent is loaded with an endothelial protein C receptor (EPCR) activator and a limus drug, and the manner of loading includes but not limited to spraying and wrapping. The vascular stent provided by the present disclosure can inhibit vascular smooth muscle cells to prevent restenosis and promote functions of endothelial cells and the re-endothelialization process of the stent to prevent stent thrombosis by loading the EPCR activator and the limus drug, and has dual effects. Moreover, when the EPCR activator used in the present disclosure relieves the inhibition effect on the endothelial cells and an endothelialization process caused by the limus drug, the drug effect of the limus drug is not affected.

### BRIEF DESCRIPTION OF THE DRAWINGS

FIG. 1 shows concentration-dependent inhibition of expression of an endothelial protein C receptor (EPCR) by rapamycin in human coronary artery endothelial cells (HCAECs).

FIG. 2 shows concentration-dependent inhibition of expression of an EPCR by zotarolimus in HCAECs.

FIG. 3 shows concentration-dependent inhibition of expression of an EPCR by everolimus in HCAECs.

FIG. 4 shows concentration-dependent inhibition of expression of an EPCR by paclitaxel in HCAECs.

FIG. 5 shows inhibition of expression of an EPCR by DMSO in a mouse heart vessel.

FIG. 6 shows inhibition of expression of an EPCR by rapamycin in a mouse cardiac vessel

FIG. 7 shows inhibition of expression of an EPCR by paclitaxel in a mouse cardiac vessel.

FIG. 8 shows inhibition of expression of an EPCR by everolimus in a mouse cardiac vessel.

FIG. 9 shows inhibition of expression of an EPCR by zotarolimus in a mouse cardiac vessel.

FIG. 10 shows inhibition of expression of an EPCR by tacrolimus, deforolimus, biolimus A9, and pimecrolimus in HCAECs.

FIG. 11 shows relative expression of EPCR protein increased by activated protein C, TR47 and Parmodulin 2 in HCAECs, and activation of an EPCR pathway.

FIG. 12 shows that activated protein C, TR47, and Parmodulin 2 can reverse rapamycin-induced EPCR protein decline and EPCR pathway inhibition caused by rapamycin in HCAECs.

FIG. 13 shows that an EPCR is expressed in endothelial cells such as human umbilical vein endothelial cells (HUVECs), human aortic endothelial cells (HAECs) and human cardiac microvascular endothelial cells (HCMECs), but almost not expressed in human aortic smooth muscle cells (HASMCs) and human coronary artery smooth muscle cells (HCASMCs).

FIG. 14 shows that overexpression of an EPCR does not affect cell viability of smooth muscle cells.

FIG. 15 shows that activation of an EPCR by APC does not affect cell viability of smooth muscle cells.

FIG. 16 shows that overexpression of an EPCR does not affect the proliferation of smooth muscle cells.

FIG. 17 shows that activation of an EPCR by APC does not affect the proliferation of smooth muscle cells.

FIG. 18 shows that overexpression of an EPCR does not affect Transwel experimental migration of smooth muscle cells.

FIG. 19 shows that activation of an EPCR by APC does not affect Transwel experimental migration of smooth muscle cells.

FIG. 20 shows that overexpression of an EPCR does not affect scratch experimental migration of smooth muscle cells.

FIG. 21 shows that activation of an EPCR by APC does not affect scratch experimental migration of smooth muscle cells.

FIG. 22 shows influence of a solvent control (tetrahydrofuran (THF) on HCAECs in a stent simulation environment.

FIG. 23 shows influence of rapamycin on HCAECs in a stent simulation environment.

FIG. 24 shows influence of APC/rapamycin on HCAECs in a stent simulation environment.

FIG. 25 shows influence of parmodulin 2/rapamycin on HCAECs in a stent simulation environment.

FIG. 26 shows influence of TR47/rapamycin on HCAECs in a stent simulation environment.

FIG. 27 shows that EPCR activators including activated protein C, TR47, and Parmodulin 2 protectively protect against the cell viability of HCAECs damaged by rapamycin.

FIG. 28 shows that EPCR activators including activated protein C, TR47, and Parmodulin 2 protectively protect against the cell proliferation capacity of HCAECs damaged by rapamycin.

FIG. 29 shows that EPCR activators including activated protein C, TR47, and Parmodulin 2 protect against cell migration capacity of HCAECs damaged by rapamycin.

FIG. 30 shows that EPCR activators including activated protein C, TR47 and Parmodulin 2 protectively inhibit adhesion of platelets to HCAECs.

FIG. 31 shows that EPCR activators including activated protein C, TR47 and Parmodulin 2 protectively inhibit adhesion of neutrophils to HCAECs.

FIG. 32 is a macroscopic shape of a representative vascular metal stent with a scale: 2 mm.

FIG. 33 is a representative scanning electron microscope image of a representative bare metal stent.

FIG. 34 is a representative scanning electron microscope image of a rapamycin-coated stent.

FIG. 35 shows that a coating with EPCR activators, including activated protein C, TR47 and Parmodulin 2 promote endothelialization of a stent in vitro.

FIG. 36 is a representative scanning electron microscope image of a BMS in vivo.

FIG. 37 is a representative scanning electron microscope image of a rapamycin-coated metal stent in vivo.

FIG. 38 is a representative scanning electron microscope image showing that an APC/rapamycin-coating promotes re-endothelialization of a metal stent and inhibition of platelet adhesion in vivo.

FIG. 39 is a representative HE staining image of arterial restenosis after BMS implantation in vivo.

FIG. 40 is a representative HE staining image of arterial restenosis after rapamycin-coated stent implantation in vivo.

FIG. 41 is a representative HE staining image of arterial restenosis after APC/rapamycin-coated stent implantation in vivo.

FIG. 42 shows intimal stenosis rates of arterial restenosis after different stent implantation.

FIG. 43 is an optical coherence tomography image 45 d after BMS implantation in abdominal aortas of rabbits.

FIG. 44 is a stent marking image of an optical coherence tomography image 45 d after BRS implantation in abdominal aortas of rabbits.

FIG. 45 is an optical coherence tomography image 45 d after rapamycin BRS implantation in abdominal aortas of rabbits.

FIG. 46 is a stent marking image of an optical coherence tomography image 45 d after rapamycin BRS implantation in abdominal aortas of rabbits.

FIG. 47 is an optical coherence tomography image 45 d after APC/rapamycin BRS implantation in abdominal aortas of rabbits.

FIG. 48 is a stent marking image of an optical coherence tomography image 45 d after APC/rapamycin BRS implantation in abdominal aortas of rabbits, showing that APC/rapamycin promotes an endothelialization process of the bioresorbable stent in vivo.

FIG. 49 shows neointimal endothelial coverage for different BRSs.

FIG. 50 shows the count of different BRSs.

FIG. 51 shows Evans blue staining of BRS arterial vessel injury with a scale: 1 mm.

FIG. 52 shows Evans blue staining of rapamycin BRS arterial vessel injury with a scale: 1 mm.

FIG. 53 shows Evans blue staining of APC/rapamycin BRS arterial vessel injury with a scale: 1 mm.

FIG. 54 shows Evans blue staining positive area rates of different BRS arterial injury.

FIG. 55 is a CD31 positive fluorescence image of BRS arteries.

FIG. 56 is an enlarged view of FIG. 55 with a scale: 0.8 mm.

FIG. 57 is a CD31 positive fluorescence image of rapamycin BRS arteries, where arrows in FIG. 58 indicate stent segments that are not completely reendothelialized in a rapamycin BRS group.

FIG. 58 is an enlarged diagram of FIG. 57 with a scale: 0.8 mm.

FIG. 59 is a CD31 positive fluorescence image of APC/rapamycin BRS arteries.

FIG. 60 is an enlarged diagram of FIG. 59 with a scale: 0.8 mm, which demonstrates that APC/rapamycin promotes an endothelialization process of the bioresorbable stent in vivo.

FIG. 61 shows positive rates of CD31 in different BRS arteries.

FIG. 62 is a scanning electron microscope image of re-endothelialization of a BRS in a rabbit abdominal aorta.

FIG. 63 is an enlarged diagram of FIG. 62, where n=11, and a scale is: 0.5 mm.

FIG. 64 is a scanning electron microscope image of re-endothelialization of a rapamycin BRS in a rabbit abdominal aorta.

FIG. 65 is an enlarged diagram of FIG. 64, where n=10, and a scale is: 0.5 mm.

FIG. 66 is a scanning electron microscope image of re-endothelialization of an APC/rapamycin BRS in a rabbit abdominal aorta.

FIG. 67 is an enlarged diagram of FIG. 66, where arrows indicate incomplete endothelialization and platelet adhesion, n=6, and a scale is: 0.5 mm.

FIG. 68 shows neointimal endothelial coverage for different BRSs.

“*” in the above figures represent P<0.05, “**” in the above figures represent P<0.01, “***” in the above figures represent P<0.001, and n.s. in the above figures represent that comparison is not statistically significant.

### DETAILED DESCRIPTION OF THE EMBODIMENTS

The present disclosure provides a vascular stent. The vascular stent is loaded with activated protein C and a limus drug, and the manner of loading includes but not limited to spraying and wrapping.

In the present disclosure, an endothelial protein C receptor (EPCR) activator is one or more of activated protein C, TR47 and Parmodulin 2. All activated protein C, TR47 and Parmodulin 2 can activate the EPCR. As a ligand and natural activator of the EPCR, activated protein C (APC) can exert functions similar to the EPCR: anticoagulation, anti-inflammation, anti-apoptosis, stabilization of an endothelial barrier and promotion of angiogenesis⁹⁻¹¹. Furthermore, coated stents using the APC alone also have an anticoagulant effect¹²⁻¹³. According to the present disclosure, it is discovered that the APC not only can activate an EPCR pathway inducing human coronary artery endothelial cell (HCAECs) protectively, induce protein expression of the EPCR, but also can antagonize the inhibitory effect of limus drugs (such as rapamycin) on the EPCR.

In the present disclosure, the limus drug optionally includes one or more of rapamycin, everolimus, zotarolimus, tacrolimus, deforolimus, biolimus A9 and pimecrolimus, more optionally rapamycin.

In the present disclosure, the spraying includes but not limited to spraying a coating of an EPCR activator and a limus drug on a metal stent, and the wrapping includes but not limited to wrapping the EPCR activator and the limus drug in a bioresorbable stent. According to the present disclosure, aiming at the characteristics of different stents, different loading forms are employed to realize the slow release of the EPCR activator and the limus drug.

In the present disclosure, spraying amounts of the EPCR activator and the limus drug are 1 μg/mm-100 μg/mm and 1 μg/mm-100 μg/mm respectively, more optionally 10 μg/mm-12 μg/mm. According to the present disclosure, aiming at the characteristics of the sprayed stent and the stent capable of being sprayed, by means of reasonable use amount proportion of the EPCR activator and the limus drug, the limus drug not only can effectively play a role, but also can the inhibition effect on endothelial cells and an endothelialization process caused by the limus drug can be relieved, and the risk of stent thrombus (ST) after vascular intervention can be reduced.

In the present disclosure, the wrapping amounts of the EPCR activator and the limus drug during wrapping are 1 μg/mm-100 μg/mm and 1 μg/mm-100 μg/mm respectively, more Optionally 10 μg/mm-12 μg According to the present disclosure, aiming at the characteristics of the wrapped stent and the stent capable of being wrapped, the use amount of the EPCR activator and the limus drug is reasonably proportioned, such that the release amount of the two substances in a degradation process reaches balance.

The present disclosure further provides a manufacturing process for the vascular stent mentioned in the above technical solution. The manufacturing process includes the following steps:

S1, perform stent pretreatment:

S2, prepare an EPCR activator-limus drug solution: dissolving the limus drug and the EPCR activator in a tetrahydrofuran solution containing 10% of poly(lactic-co-glycolic acid) (PLGA); and

S3, perform rotary spraying.

In the present disclosure, final concentrations of the limus drug and the EPCR activator in S2 are both 50 μg/mL.

In order to better understand the present disclosure, the contents of the present disclosure are further clarified in combination with examples below, but the contents of the present disclosure are not limited to the following examples. The materials, reagents, etc. used in the examples and test examples of the present disclosure are commercially available unless otherwise specified, and the methods used in the examples and the test examples of the present disclosure are conventional unless otherwise specified.

Embodiment 1 EPCR activators promote an endothelialization process without affecting vascular smooth muscle cells

### 1.1 Materials

APC: Sigma-Aldrich Cat #P2200;
Rapamycin: MedChemExpress Cat #HY-10219;
Metal stent: provided by Liaoning Yinyi Biotechnology Co., Ltd.;
Bioresorbable stent: provided by Hua'an Biotechnology XINSORB® (Shandong, China);
New Zealand rabbits: provided by Animal Center of Guangdong Province; and
Human coronary artery endothelial cells (HCAECs): purchased from Cell Applications.

### 1.2 Limus and Paclitaxel can Inhibit Expression of an EPCR in Vascular Endothelial Cells

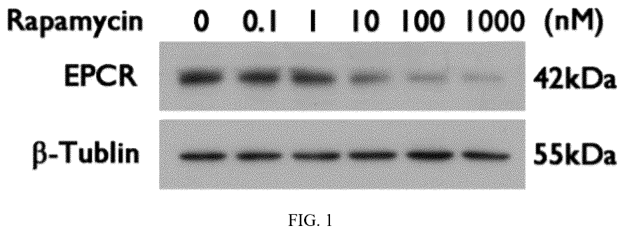

Specifically, HCAECs are cultured in an endothelial cell basal medium (EBM)-2 (Lonza, CC3156) supplemented with a 3% fetal bovine serum (FBS) and endothelial growth factor supplement mixture (Lonza, CC4176) under standard cell culture conditions (37° C., 5% CO₂), and P3-5 cells are tested at 70%-80%. Western blotting is performed to test protein expression after HCAECs are treated with rapamycin, zotarolimus, everolimus, and paclitaxel at the doses shown in FIG. 1, or 100 nM of tacrolimus, deforolimus, biolimus A9, and pimecrolimus for 24 h. Alternatively, C57BL6/J mice received rapamycin, zotarolimus, everolimus, and paclitaxel at 2 mg/kg of body weight daily via tail vein injection for 2 weeks, and immunofluorescence observation of tissues is performed.

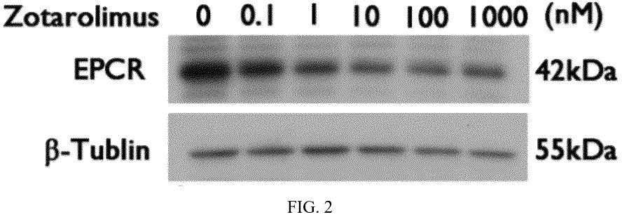

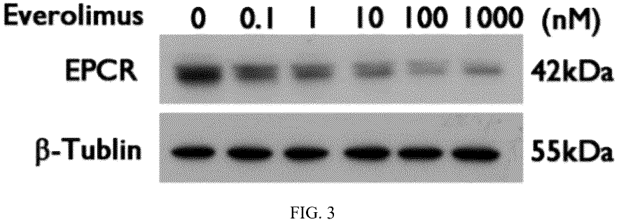

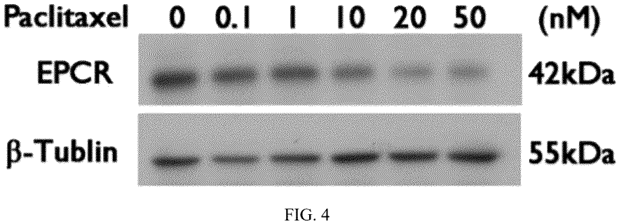

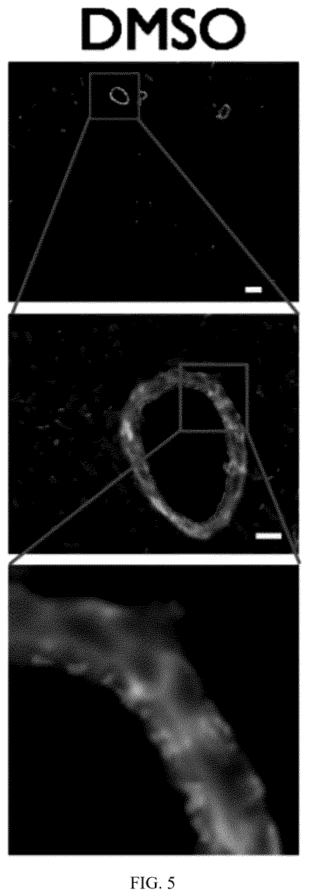

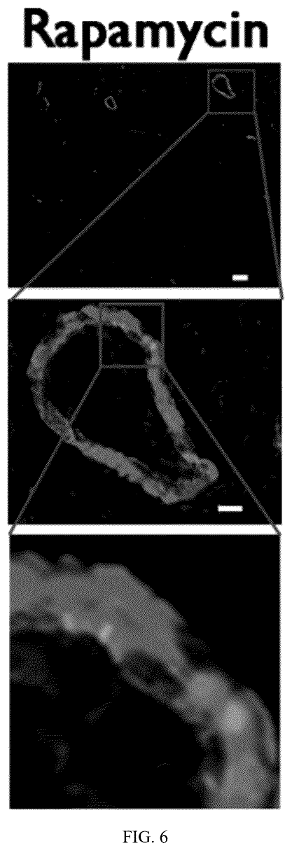

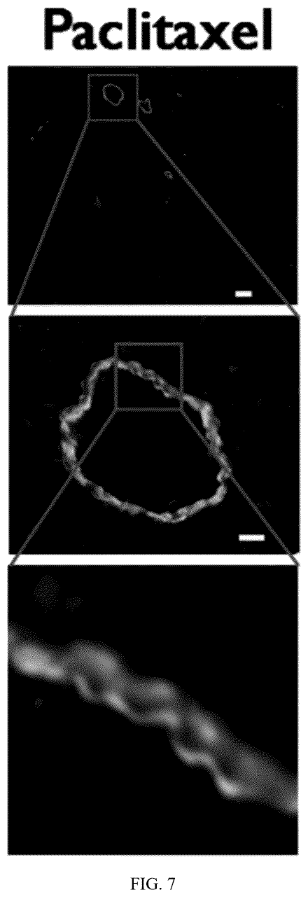

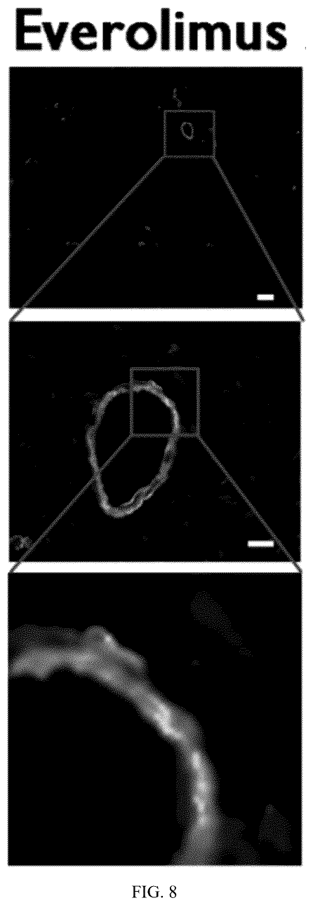

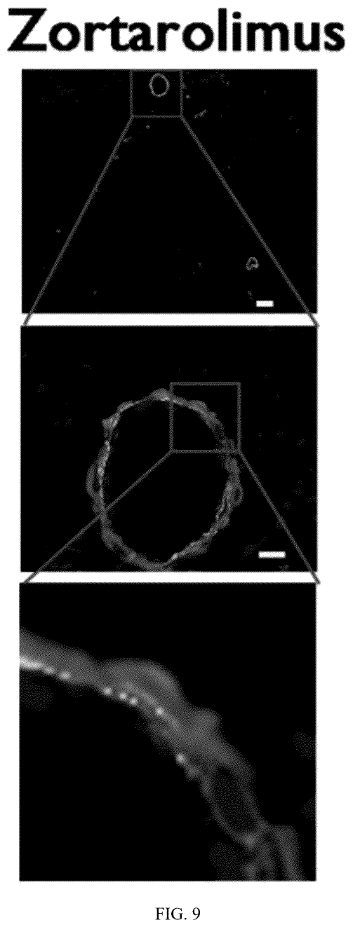

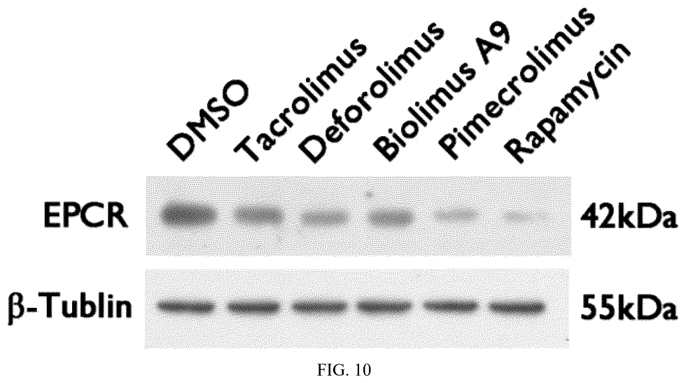

Results are shown in FIGS. 1-10. FIGS. 1-10 show that rapamycin, zotarolimus, everolimus, and paclitaxel inhibit expression of an EPCR in HCAECs in a concentration-dependent manner (FIGS. 1-4), rapamycin, zotarolimus, everolimus, and paclitaxel inhibit expression of the EPCR in mouse cardiac vessels (FIGS. 5-9), and other rapamycin derivatives including tacrolimus, deforolimus, biolimus A9, and pimecrolimus inhibit expression of the EPCR in HCAECs (FIG. 10).

### 1.3 EPCR Activators Including APC, TR47 or Parmodulin 2 Induce Expression of the EPCR in Human Coronary Artery Endothelial Cells (HCAECs)

Specifically, HCAECs are cultured in an EBM-2 (Lonza, CC3156) supplemented with a 3% fetal bovine serum (FBS) and endothelial growth factor supplement mixture (Lonza, CC4176) under standard cell culture conditions (37° C., 5% of CO₂), and cells of generations of P3-5 are tested at 70%-80%. HCAECs are treated with 10 μM of APC, 40 μM of TR47 and 20 μM of Parmodulin 2, or each in combination with 100 nM of rapamycin for 24 h, and then, western blotting is performed for testing protein expression.

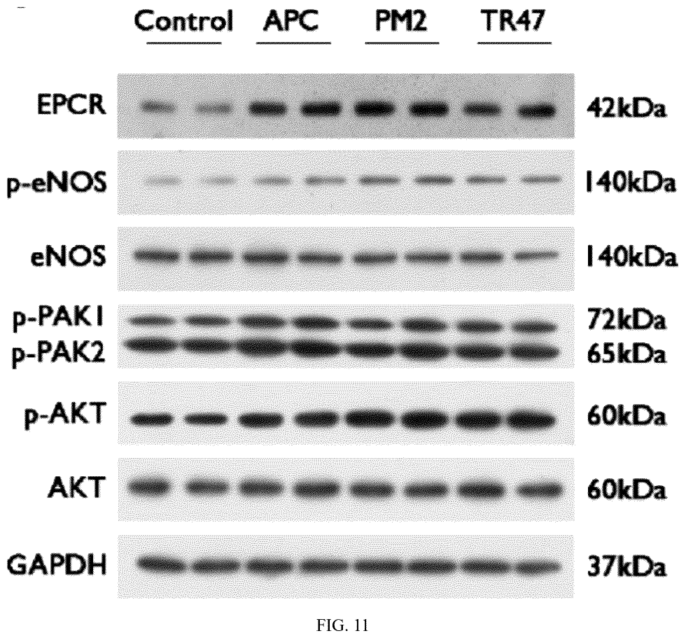

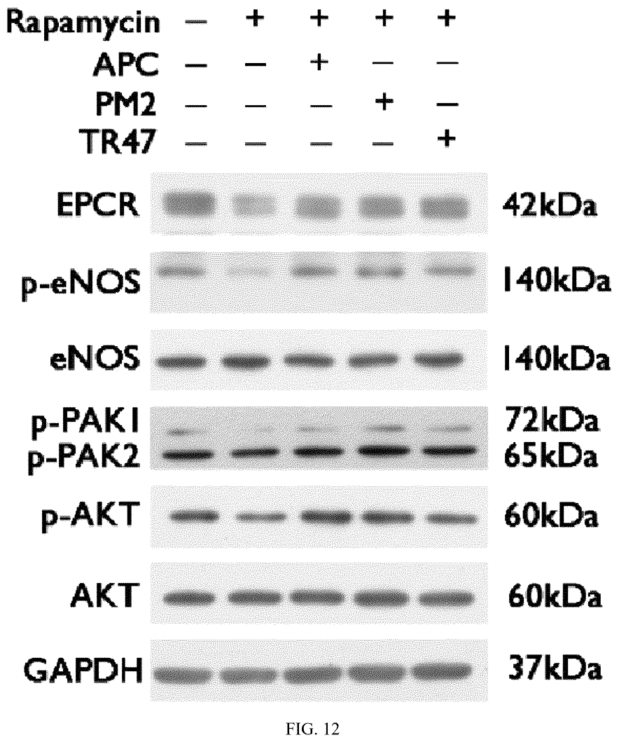

Results are shown in FIGS. 11 and 12. FIGS. 11 and 12 show that the EPCR activators including APC, TR47, or Parmodulin 2 not only protectively activate the EPCR pathway inducing HCAECs, but also induce protein expression of the EPCR in the cells (FIG. 11). The EPCR activators including APC, TR47, or Parmodulin 2 also antagonize the inhibitory effect of limus drugs such as rapamycin on the EPCR (FIG. 12).

### 1.4 Overexpression or APC Activation of EPCR does not Affect Biological Behavior of Vascular Smooth Muscle Cells and Inhibitory Effects of Rapamycin

Generations 3-5 of primary human coronary artery smooth muscle cells (HCASMCs) are cultured in a growth medium (Cell applications, cat #31-500). Human arterial smooth muscle cells (HASMCs) are cultured in a smooth muscle cell culture medium containing 2% of FBS and 1% of penicillin/streptomycin at 37° C. in a humidified atmosphere containing 5% of CO₂. Ad-EC or Ad-EPCR infected the cells for 48 h. APC is 10 μM, and rapamycin is 100 nM. Fluorescence is observed by a microscope. Protein expression is tested by means of western blotting. The cell viability is tested by means of CCK-8, the cell multiplication capacity is observed by means of EdU, and the migration capacity is evaluated by means of Transwel chamber and scratch tests.

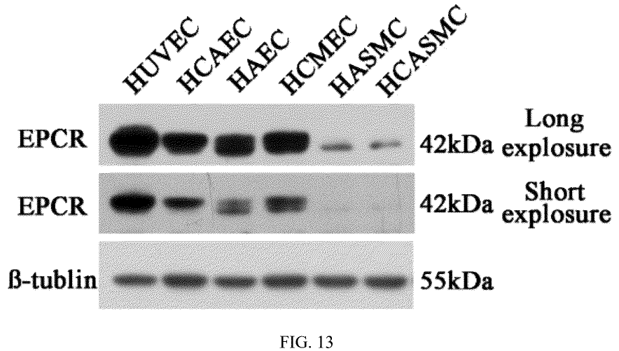

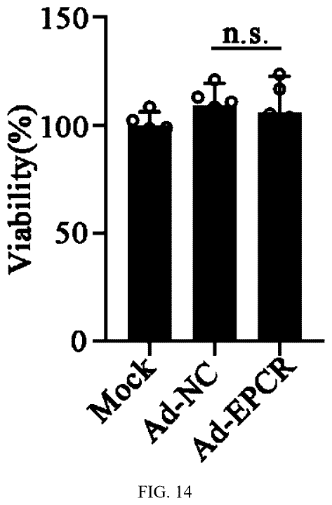

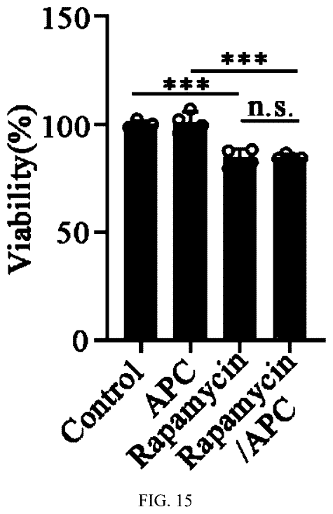

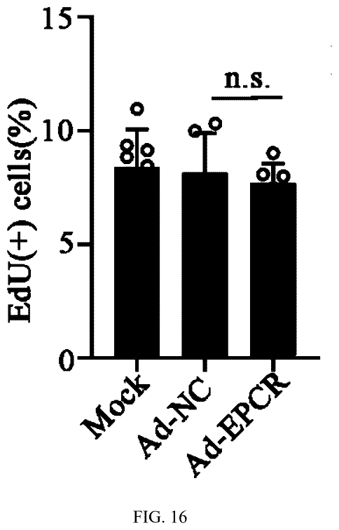

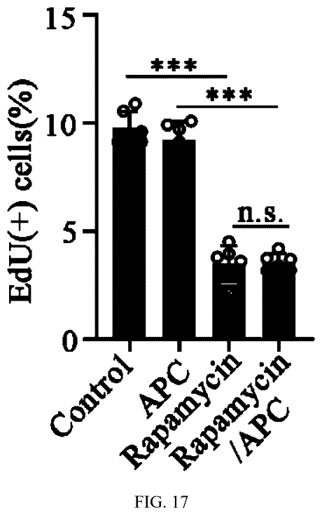

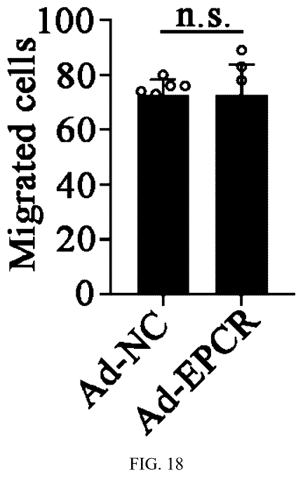

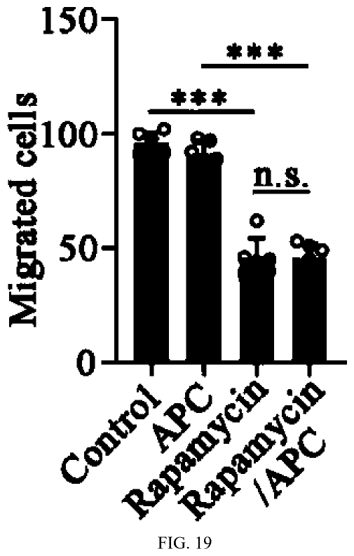

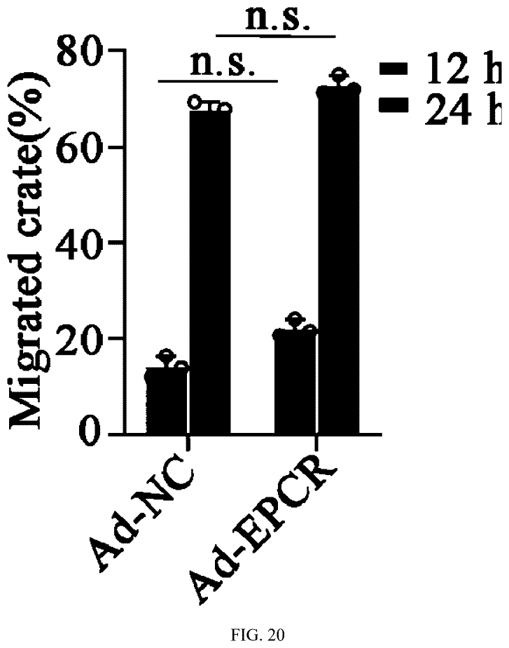

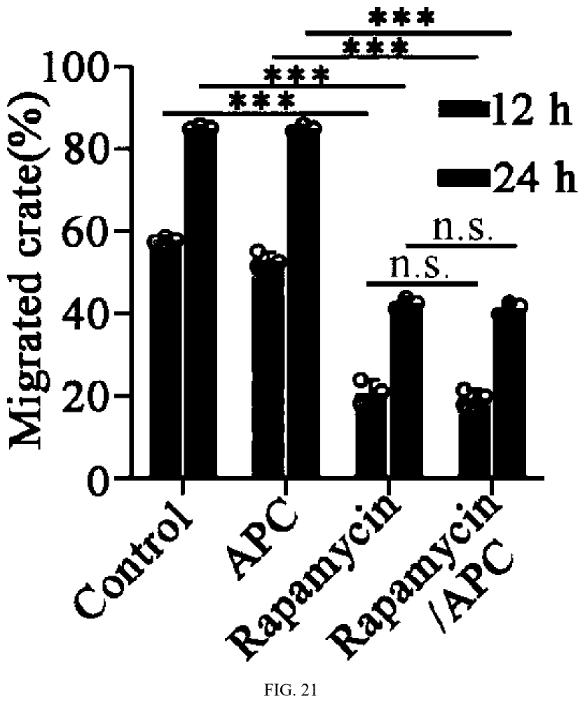

Results are shown in FIGS. 13-21. FIGS. 13-21 show that the EPCR is expressed in endothelial cells such as human umbilical vein endothelial cells (HUVECs), HCAECs, human aortic endothelial cells (HAECs) and human cardiac microvascular endothelial cells (HCMECs), but almost not expressed in human aortic smooth muscle cells (HASMCs) and human coronary artery smooth muscle cells (HCASMCs) (FIG. 13). Overexpression or APC activation of the EPCR does not affect the cell viability (FIGS. 14 and 15), the multiplication capacity (FIGS. 16 and 17) and the migration capacity (FIGS. 18-21) of the vascular smooth muscle cells.

### 1.5 Activators Including APC, TR47 and Parmodulin 2 of the EPCR Promote an Endothelialization Process in a Stent Simulation Environment

The effect of APC on a simulated stent environment of PLGA slow-release coating on a 316 L steel sheet is investigated. Slow-release coatings of a solvent control (tetrahydrofuran, THF) wrapped in poly(lactic-co-glycolic acid) (PLGA 75:25), rapamycin (50 μg/mL), APC (50 μg/mL), 40 μM of TR47, and 20 μM of Parmodulin 2, or each in combination with rapamycin (50 μg/mL) are coated on surfaces of 316 L stainless steel plates respectively.

After HCAECs are grown on the 316 L stainless steel plate for 48 h, the cell viability is tested by a CCK-8 method, the cell multiplication capacity is tested by an EdU method, and the cell migration capacity is tested by a scratch method. The HCAECs are co-cultured with neutrophils (10⁶/well) or platelets (5×10⁶/well) labeled with 2 μM of PKH-26 for 1 h. Calcein-AM staining is followed by fluorescence microscopy.

The above cells are inoculated on 316 SS, 316 L SS/Rapamycin, 316 L SS/Rapamycin/APC, 316 L SS/Rapamycin/TR47, and 316 L SS/Rapamycin/Parmodulin 2 in a 24-well plate at a density of 2×10⁴ cells/mL. 10 μL of CCK-8 reaction solution is added to a 24-well plate according to CCK-8 instructions, and the optical density value (OD value) is measured at 4 h by a spectrophotometer with a wavelength of 450 nm. For EdU analysis, reagent A is added a medium in a 24-well plate and cultured at 37° C. for 4 h to label EdU, and then, cells are captured with a fluorescence microscope (EVOS FL) according to manufacturer's instructions. Cell adhesion is examined by means of staining with Calcein-AM (5 μM) for 1 h.

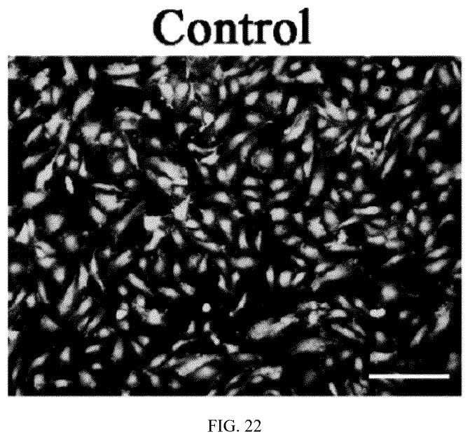

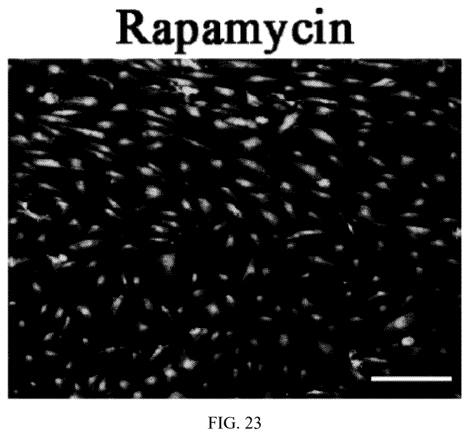

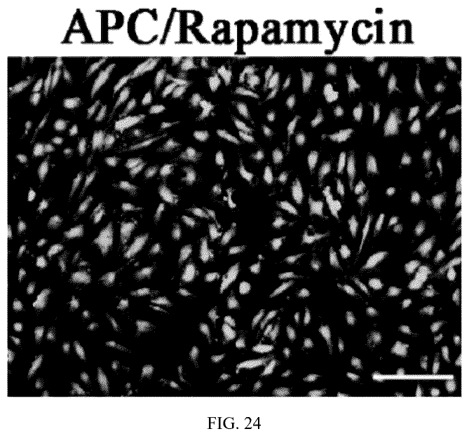

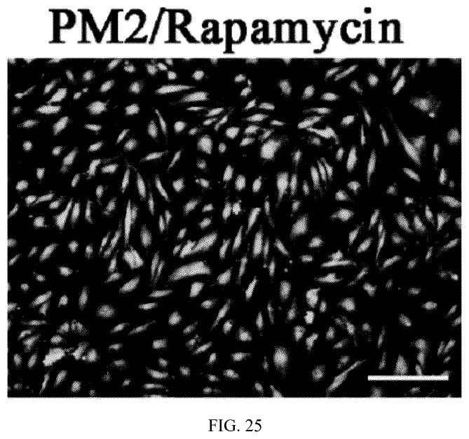

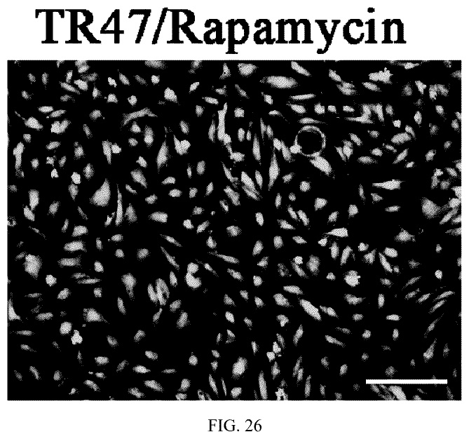

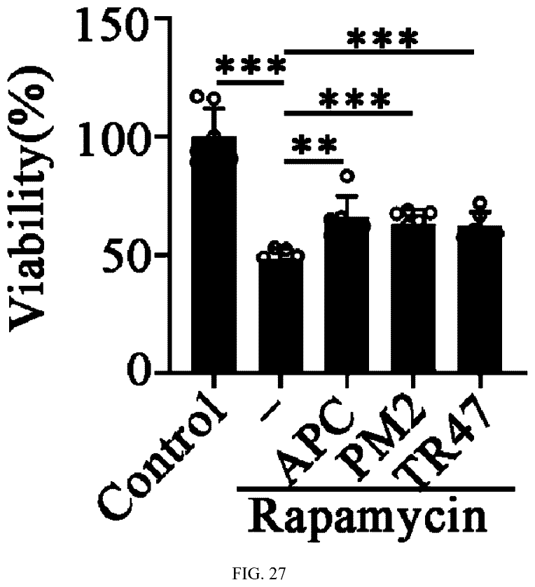

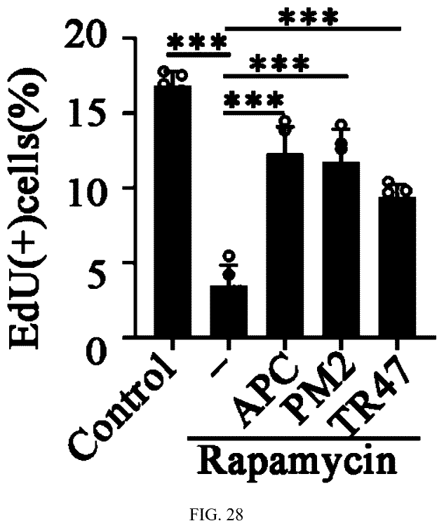

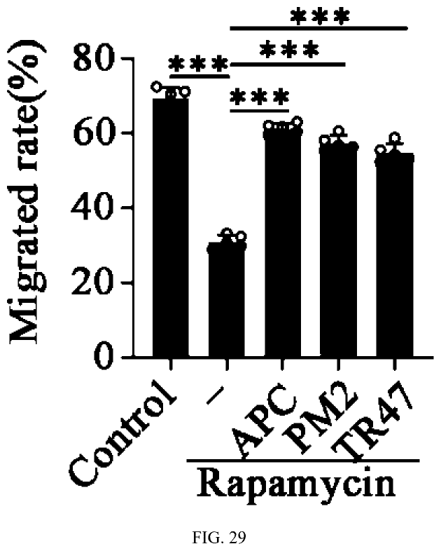

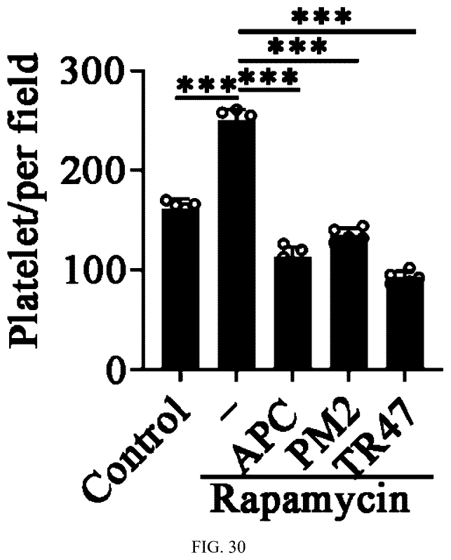

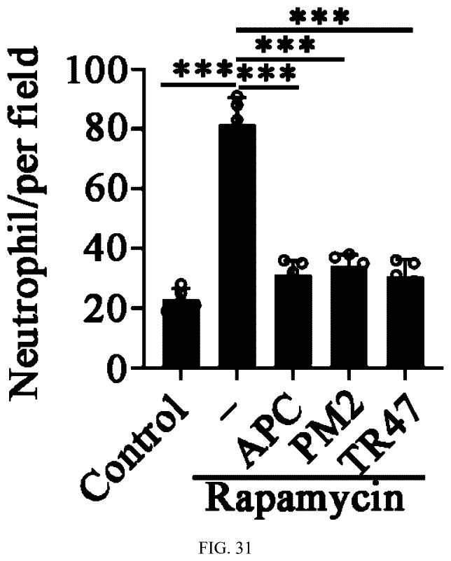

Results show that PLGA-rapamycin inhibits the growth, viability, multiplication, and migration of the HCAECs on the surfaces of the 316 L stainless steel (FIGS. 22-29) and promote adhesion of platelets (FIG. 30) and neutrophils (FIG. 31) to surface of ECs. In contrast, the slow-release coating using activators of EPCR including APC, TR47, or Parmodulin 2 in combination with rapamycin can restore the growth, viability, multiplication, and migration of the HCAECs (FIGS. 22-29) and inhibit adhesion of the platelets and neutrophils (FIGS. 30 and 31).

Embodiment 2 Vascular metal stent

### 2.1 Manufacturing of Vascular Stent (Spraying)

The vascular stent is a metal stent coated with activated protein C and rapamycin. The steps include:

(1) Perform stent pretreatment: a clinical-grade 316 L bare metal stent (2.5 mm×15 mm, diameter×length) is treated by means of conventional laser cutting and sterilization, etc.

(2) Prepare activated protein C/rapamycin, 40 μM of TR47/rapamycin, and 20 μM of Parmodulin/rapamycin solutions: 50 μg/mL of rapamycin and 50 μg/mL APC or 40 μM of TR47 or 20 μM of Parmodulin are dissolved in a tetrahydrofuran solution containing 10% of PLGA.

(3) Perform rotary spraying: The clinical-grade 316 L bare metal stent and a bioresorbable stent are coated with approximately 12 μg/mm of rapamycin and 10 μg/mm of APC, TR47 or Parmodulin by using a rotary spraying technology.

Briefly, the poly(lactic-co-glycolic acid) (PLGA 75:25) is encapsulated and dissolved in tetrahydrofuran (THF) at a concentration of 10% (w/v) and stirred at a room temperature for 12 h for later use. 12 μg/mL of rapamycin and/or 10 μg/mL of APC is then added and dissolved for 1 h. 2 mL of PLGA/Rapamycin or PLGA/Rapamycin/APC solution is pumped by a syringe pump at speeds of 1 mL/h and 0.1 mL/h respectively, a high voltage of 15 kV is applied, and a collection distance is fixed at 15 cm. The same volume of PLGA solution but without the rapamycin and the APC is used as a control. In this experiment, all coatings are collected on a rotating drum for 1 h and then dried in vacuum at 40° C. for more than 12 h to remove residual solvents for further use.

### 2.2 Endothelialization Test of Stent In Vitro

A bare metal stent and a rapamycin-coated metal stent are used as controls (manufacturing processes are as described above). After the HCAECs are co-cultured with the bare metal stent, the rapamycin-coated metal stent and the APC+rapamycin-coated metal stent for 3 d, endothelial cells are stained with 5 μM of calcein AM (green) and tested by a fluorescence microscope.

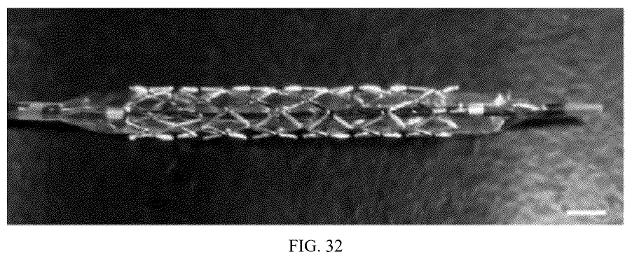

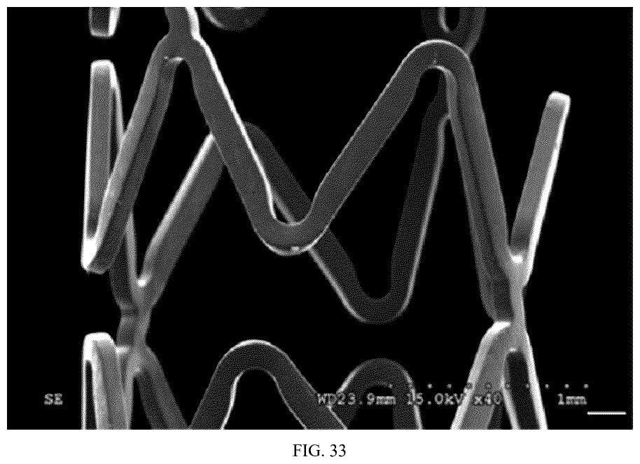

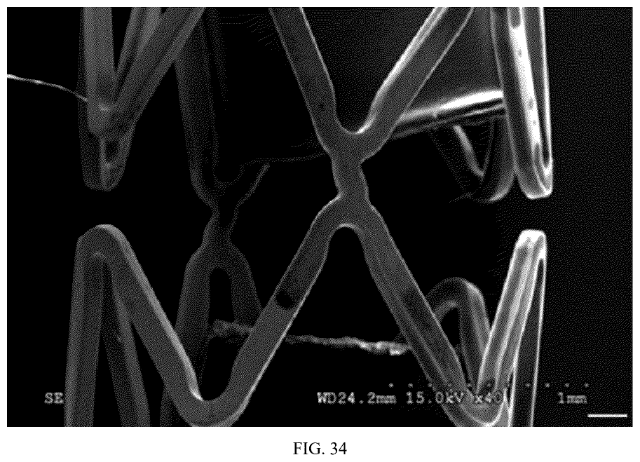

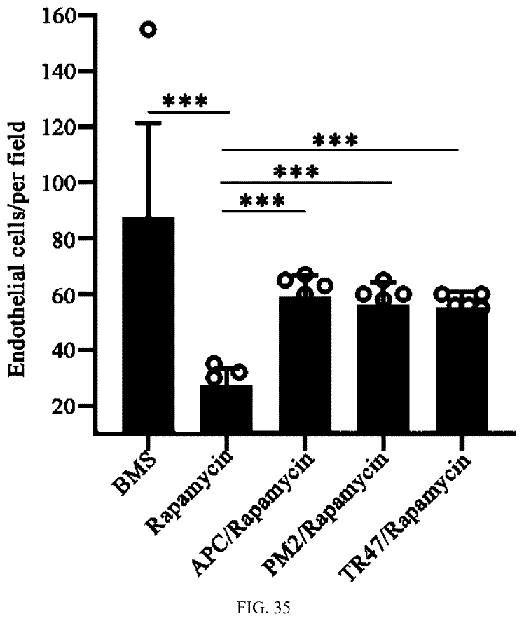

Results are shown in FIGS. 32-35. FIGS. 32-35 show that the endothelial cell count of the rapamycin stent is significantly lower than that of the bare metal stent, while the endothelial cell counts of APC+rapamycin, TR47+rapamycin, Parmodulin 2+rapamycin-coated metal stents are significantly higher than that of the rapamycin stent, indicating that the EPCR activators including APC, TR47 and Parmodulin 2 can significantly relieve the inhibitory effect of rapamycin on the endothelial cells. The APC+rapamycin coating can promote the endothelialization process of the stent in vitro.

### 2.3 Endothelialization Test of Stent In Vivo

Animals used in the present disclosure are healthy new Zealand white rabbits (3.0 kg-3.5 kg), which are placed in individual cages in a temperature and light control room, and standard rabbit food and sterilize drinking water are provided free of charge. All experimental procedures complied with the Guidelines for the Care and Use of Laboratory Animals issued by the National Institutes of Health (NIH Publication No. 85-23, revised in 1996) of the United States and approved by the Animal Ethics Committee of Guangzhou Medical University. Briefly, the rabbits received aspirin at 2 mg/kg and clopidogrel at 1.5 mg/kg orally daily prior to stent implantation. The rabbits are divided into three groups: bare metal stent (BMS), BMS coated with rapamycin, and BMS coated with APC+rapamycin. The rabbits are anesthetized by intravenous injection of pentobarbital sodium (30 mg/kg) into the marginal ear vein. The stents (diameter of 3.5 mm and length of 12 mm) coated with different drugs are assembled on an inflatable stent. The stent is implanted into the abdominal aorta 10 mm down from the bifurcation of the right femoral artery incision through a 6F sheath (Terumo, RS* A60K10SQ). The balloon is inflated to 12-16 ATM for 30 s, and then deflated for 30 s to maintain negative pressure. Repetition is performed three times to fully unfold the stent. Subsequently, the balloon is deflated and slowly withdrawn, leaving the stent in place for angiographic observation (GE Healthcare). The right femoral artery is sutured with a 7-0 biosoluble suture. Penicillin (5000 U/kg) is injected into all the animals within 3 days after operation. The rabbits are injected with 2-2.5 mL of heparin (250 mL 0.9% NaCl+6000 U) each time throughout the operation procedure. After 28 d, the ultrastructures of stent arteries are observed under a scanning electron microscope, and HE staining is used for analyzing arterial restenosis after stent implantation.

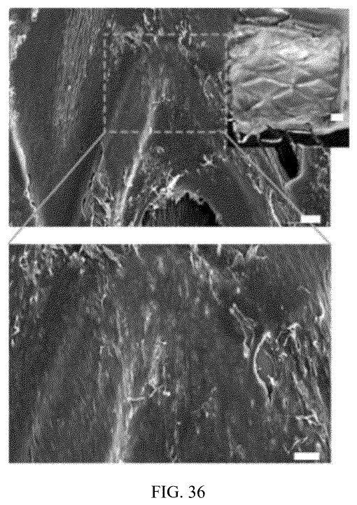

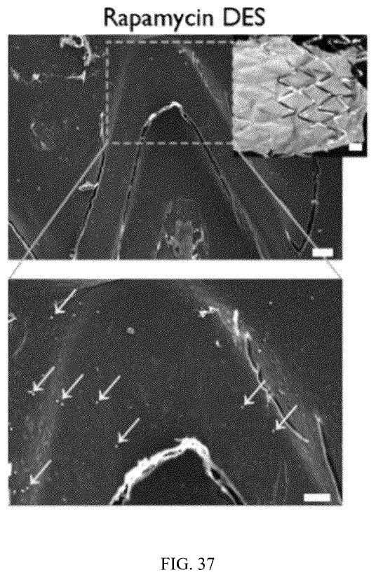

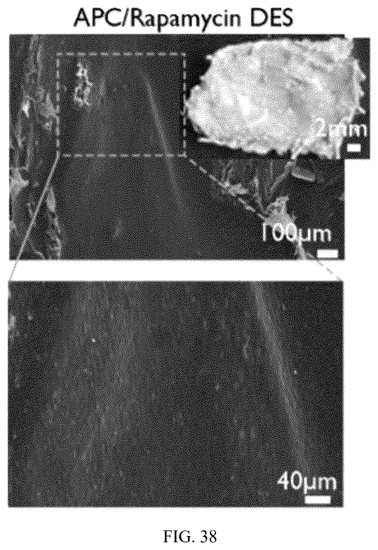

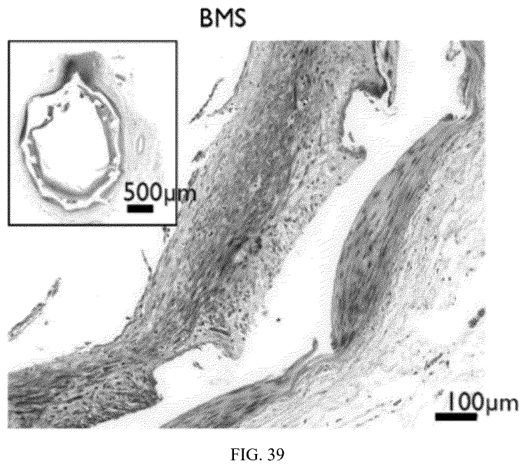

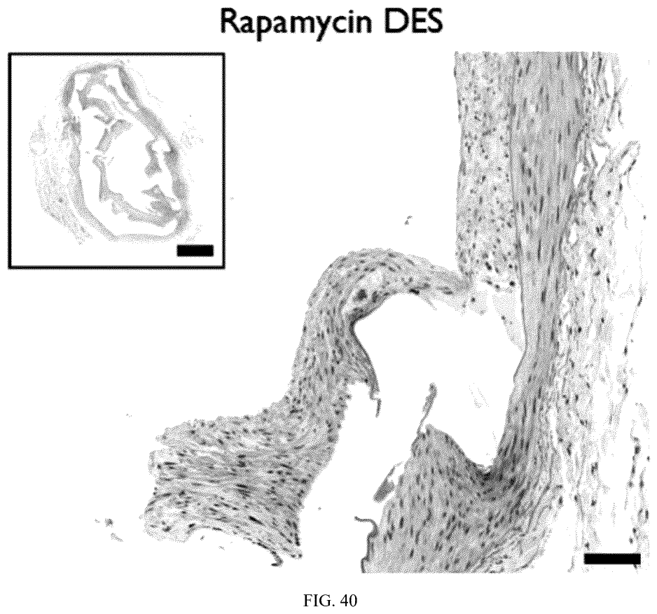

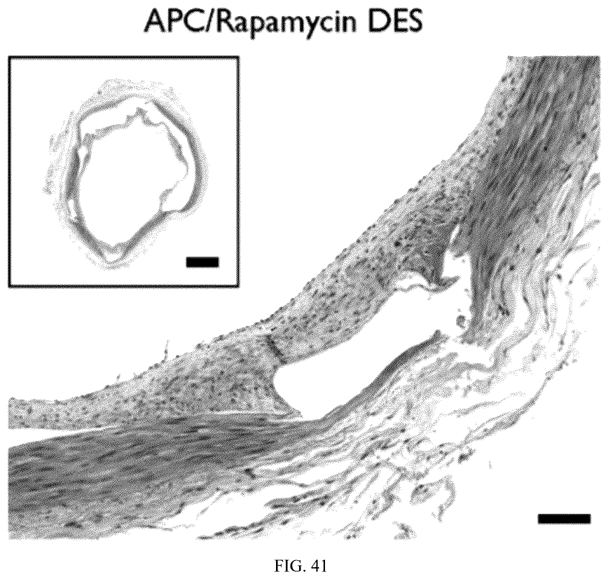

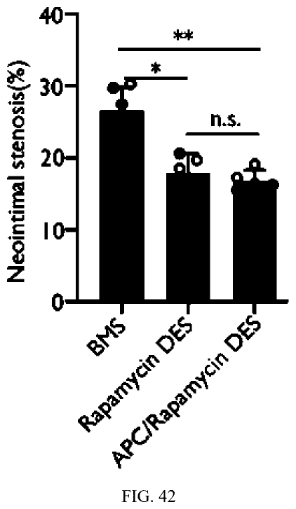

Results are shown in FIGS. 36-42. FIGS. 36-38 show that after 28 d, the ultrastructure of the arterial lumen of the stent in each group is observed under the scanning electron microscope. Compared with the rapamycin stent, the endothelial cells adhered to the surface of the APC+rapamycin stent form a uniform cobblestone-like cell layer, the stent is almost completely covered by the endothelial cells, and the main axis direction is consistent with blood flow, while the platelet adhesion on the stent surface is significantly reduced (golden arrow). It can be seen from FIGS. 39-42 that images stained with hematoxylin and eosin (HE) show that bare metal stents have more intimal hyperplasia than rapamycin and APC+rapamycin stents, and there is no significant statistical difference in intimal hyperplasia between the two after implantation, indicating that APC does not affect the effect of rapamycin on inhibiting smooth muscle cell multiplication and restenosis, that is, APC does not affect the drug effect of rapamycin, and the combination of APC and rapamycin does not conflict. Thus, the APC+rapamycin stent significantly promotes the endothelialization process of the metal stent without affecting restenosis.

### Embodiment 3 Vascular Stent in Wrapped Manner (Bioresorbable)

### 3.1 Various Customized Stents of XINSORB®

(1) Bioresorbable stent (BRS) control
(2) Rapamycin BRS
(3) APC+Rapamycin BRS

The above three types of stents are customized at Hua'an Biotechnology XINSORB® where stents (1) and (2) are bioresorbable stents (Xinsorb, model: 3.5 mm×12 mm with the rapamycin load of 12 μg/mm) manufactured by the company for clinical use, and for stent (3), rapamycin+APC is added according to our requirements in the manufacturing process of the bioresorbable stent by the company. After molding, the final content of rapamycin is 12 μg/mm, and the final content of APC is 10 μg/mm.

### 3.2 Effect of Bioresorbable Stents In Vivo

The BRS control, the rapamycin BRS, and the APC+rapamycin BRS are implanted into abdominal aortas of rabbits respectively (specific steps are the same as 2.3). After 45 d, optical coherence tomography, Evans blue staining, CD31 immunofluorescence and scanning electron microscopy are performed.

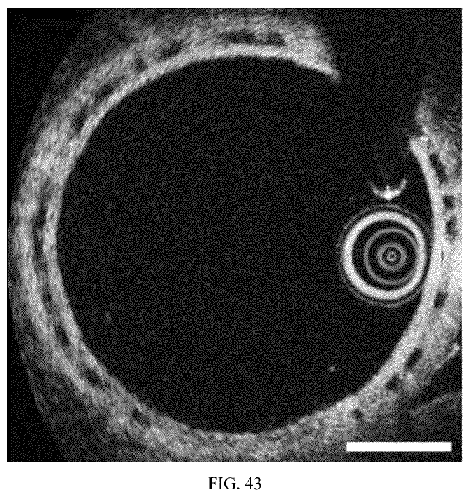

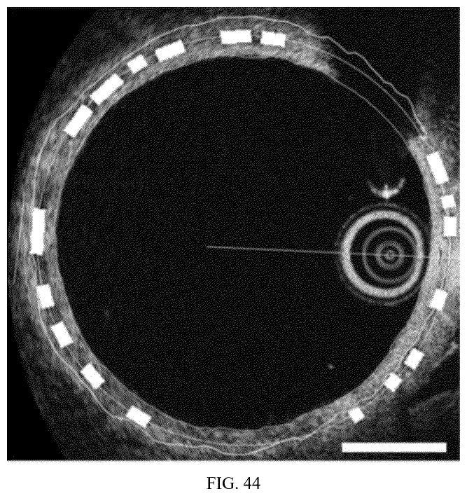

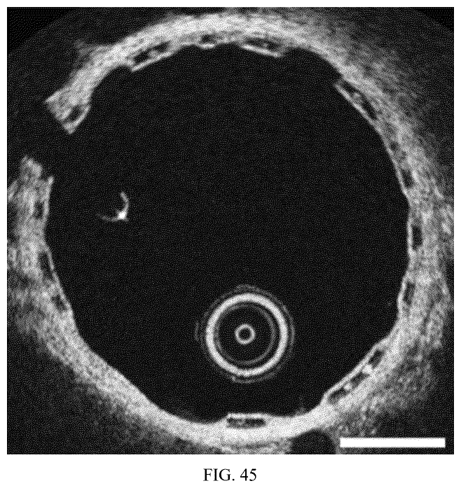

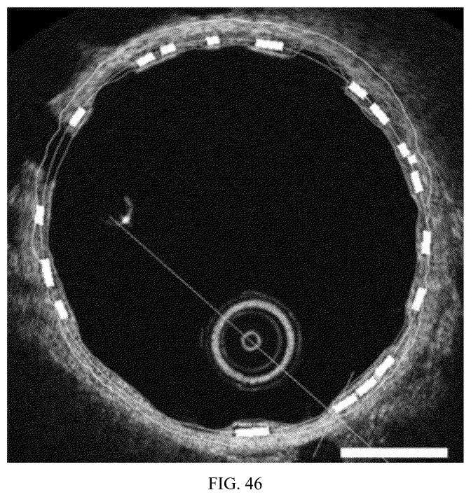

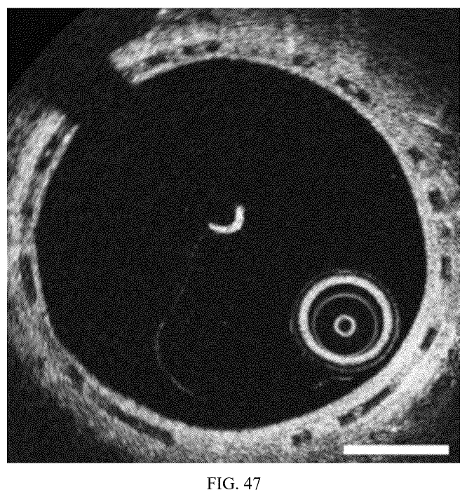

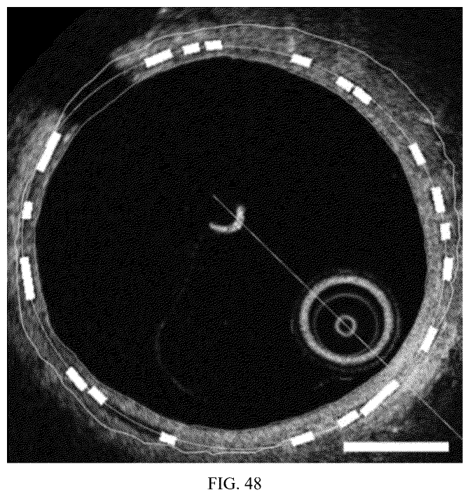

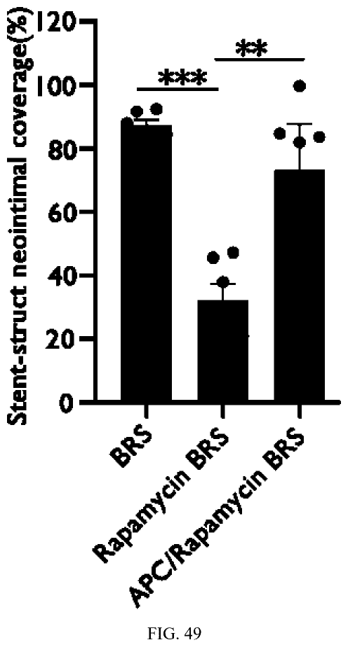

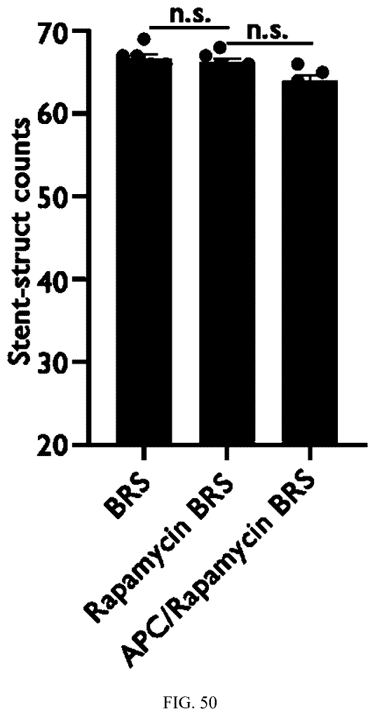

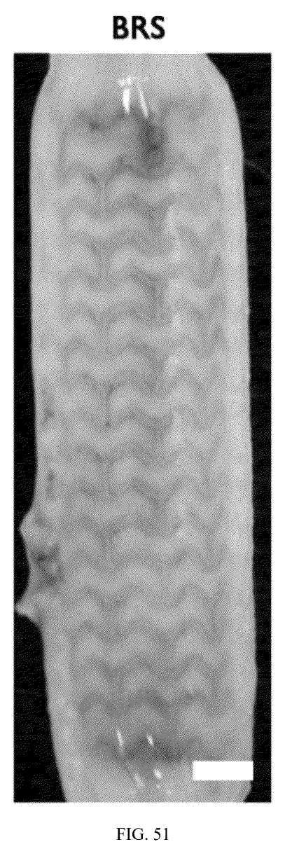

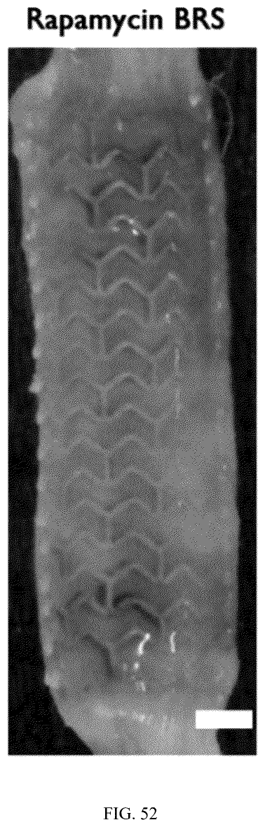

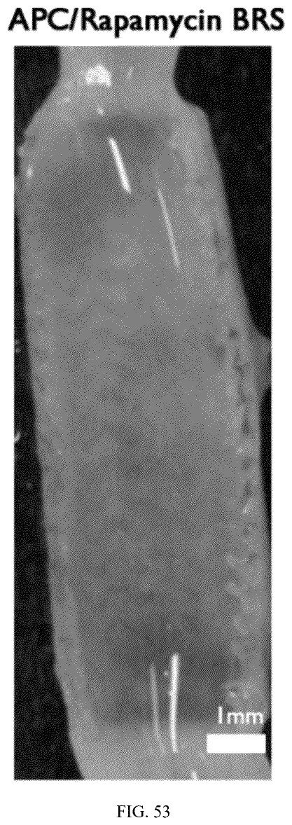

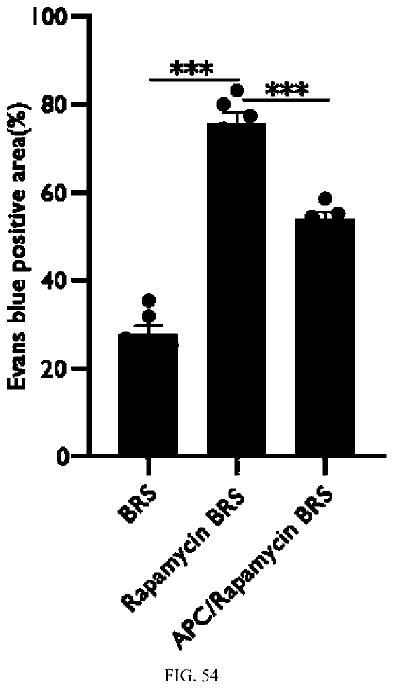

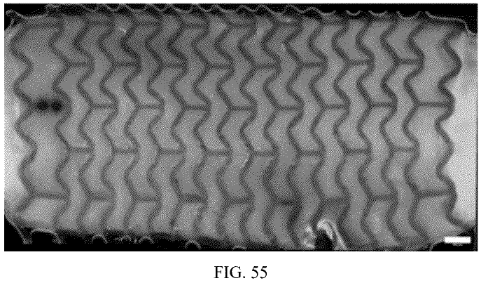

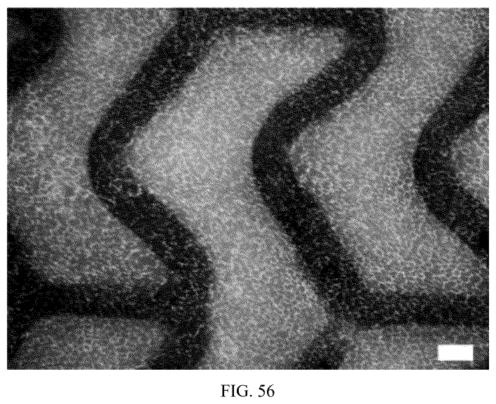

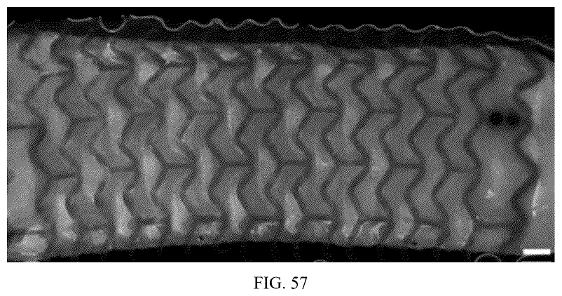

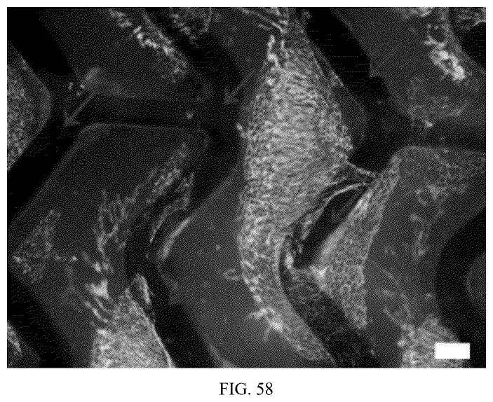

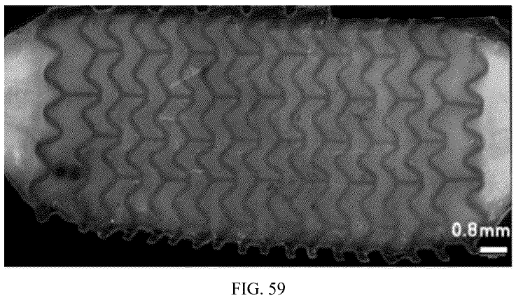

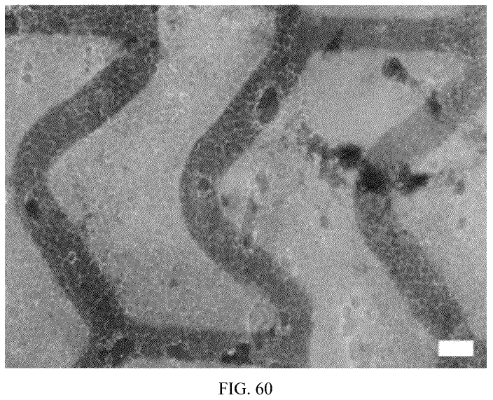

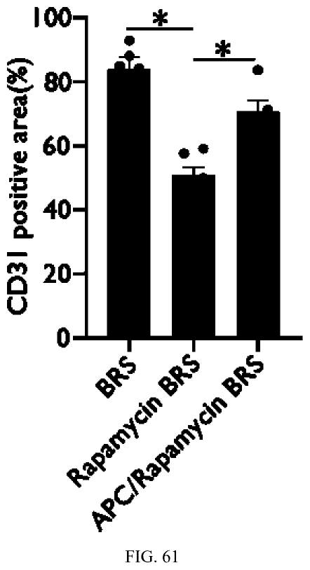

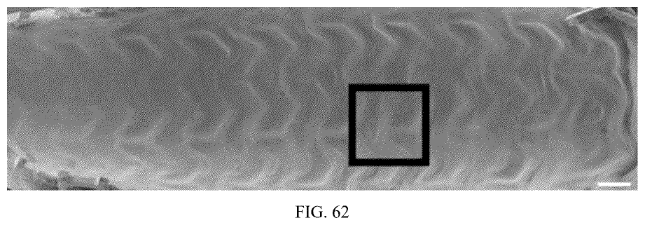

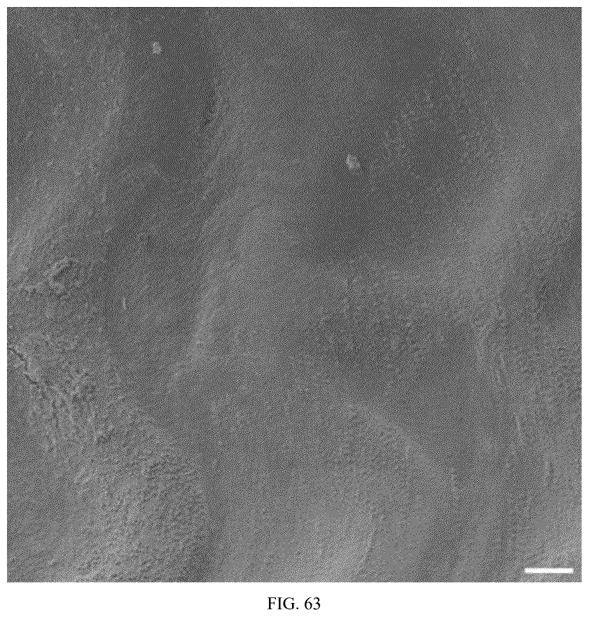

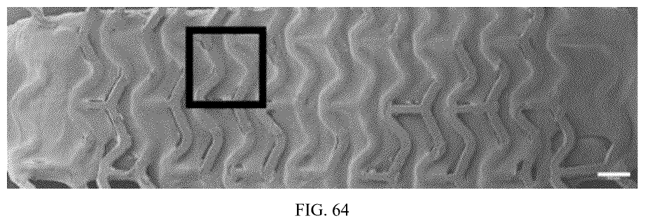

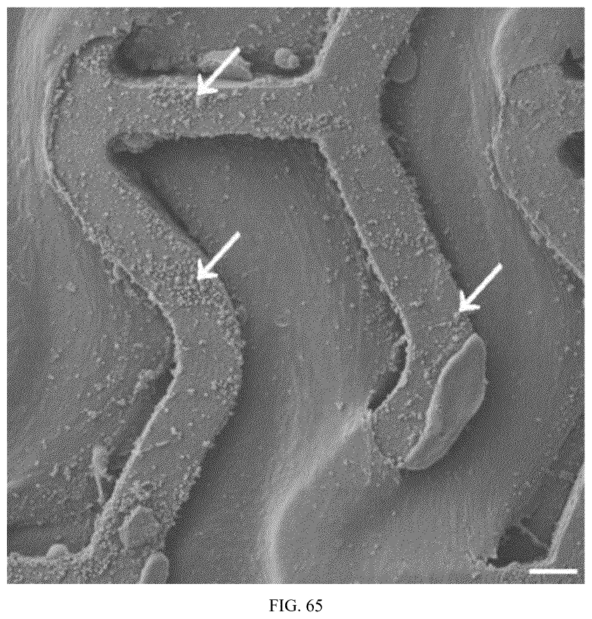

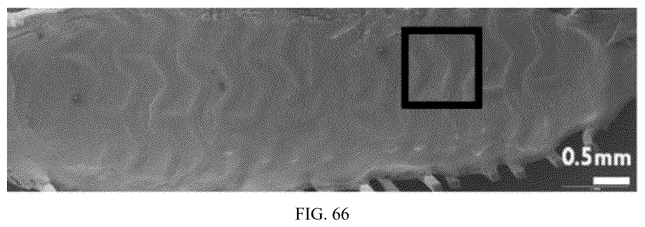

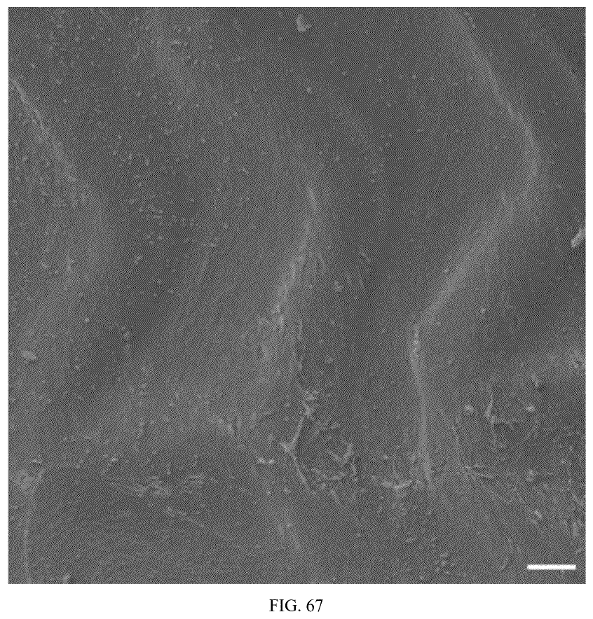

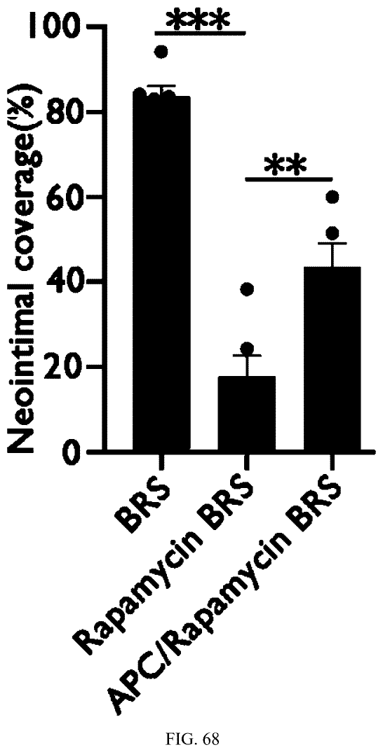

Results are shown in FIGS. 43-68. Results of the optical coherence tomography show a significant increase to intimal coverage in the APC+rapamycin BRS group (FIGS. 43-50). Results of the Evans blue staining show that the vascular injury in the APC+rapamycin BRS group is significantly better than that in the rapamycin BRS group (FIGS. 51-54). Results of CD31 immunofluorescence show that the APC+rapamycin BRS has significantly better EC coverage than the rapamycin BRS (FIGS. 55-61). Results of the scanning electron microscopy show that endothelial cells adhered to the surface of the APC+rapamycin BRS form a uniform cobblestone-like cell layer, and the main axis direction is consistent with blood flow with little platelet adhesion, and re-endothelialization is relatively complete (FIGS. 62-68). Therefore, in vivo experiments show that the APC+rapamycin bioresorbable stent can significantly promote the endothelialization process of the stent and reduce platelet adhesion.

In conclusion, the vascular stents using EPCR activators including APC, TR47 and Parmodulin 2 in combination with limus (such as rapamycin) can effectively promote the endothelialization process of the stent, reduce platelet adhesion and thrombosis, and do not affect the function of inhibiting restenosis of limus (such as rapamycin).

The above-mentioned descriptions are merely the preferred embodiments of the present disclosure, it should be pointed out that those of ordinary skill in the art can also make some improvements and modifications without departing from the principle of the present disclosure, and these improvements and modifications should also fall within the protection scope of the present disclosure.

### REFERENCES

1. Torrado J, Buckley L, Duran A, Trujillo P, Toldo S, Valle Raleigh J, Abbate A, Biondi-Zoccai G, Guzman L A. Restenosis, Stent Thrombosis, and Bleeding Complications: Navigating Between Scylla and Charybdis. J Am Coll Cardiol 2018; 71(15):1676-1695.
2. Kimura T, Morimoto T, Nakagawa Y, Kawai K, Miyazaki S, Muramatsu T, Shiode N, Namura M, Sone T, Oshima S, Nishikawa H, Hiasa Y, Hayashi Y, Nobuyoshi M, Mitudo K, j-Cypher Registry I. Very late stent thrombosis and late target lesion revascularization after sirolimus-eluting stent implantation: five-year outcome of the j-Cypher Registry. Circulation 2012; 125(4):584-91.
3. Camici G G, Steffel J, Amanovic I, Breitenstein A, Baldinger J, Keller S, Luscher T F, Tanner F C. Rapamycin promotes arterial thrombosis in vivo: implications for everolimus and zotarolimus eluting stents. Eur Heart J 2010; 31(2):236-42.
4. Wu Y, Yin J, Chen J, Yao Z, Qian J, Shen L, Ge L, Ge J. Final report of the 5-year clinical outcomes of the XINSORB bioresorbable sirolimus-eluting scaffold in the treatment of single de novo coronary lesions in a first-in-human study. Ann Transl Med 2020; 8(18):1162.
5. Yamaji K, Ueki Y, Souteyrand G, Daemen J, Wiebe J, Nef H, Adriaenssens T, Loh J P, Lattuca B, Wykrzykowska J J, Gomez-Lara J, Timmers L, Motreff P, Hoppmann P, Abdel-Wahab M, Byrne R A, Meincke F, Boeder N, Honton B, O'Sullivan C J, Ielasi A, Delarche N, Christ G, Lee J K T, Lee M, Amabile N, Karagiannis A, Windecker S, Raber L. Mechanisms of Very Late Bioresorbable Scaffold Thrombosis: The INVEST Registry. J Am Coll Cardiol 2017; 70(19):2330-2344.
6. Duarte J H. Coronary artery disease: durable-polymer drug-eluting stents might not lead to very late stent thrombosis. Nat Rev Cardiol 2015; 12(1):3.
7. Serruys P W, Revaiah P C, Onuma Y. Bioresorbable Scaffolds: Is There Still Light at the End of the Tunnel? J Am Coll Cardiol 2023; 82(3):196-199.
8. Chaus A, Uretsky B F. Bioresorbable Vascular Scaffolds: a Dissolving Dream? Cardiovasc Drugs Ther 2023; 37(1):1-3.
9. Oto J, Fernandez-Pardo A, Miralles M, Plana E, Espana F, Navarro S, Medina P. Activated protein C assays: A review. Clin Chim Acta 2020; 502:227-232.
10. Griffin J H, Zlokovic B V, Mosnier L O. Activated protein C: biased for translation. Blood 2015; 125(19): 2898-907.
11. Shahzad K, Kohli S, Al-Dabet M M, Isermann B. Cell biology of activated protein C. Curr Opin Hematol 2019; 26(1):41-50.
12. Lukovic D, Nyolczas N, Hemetsberger R, Pavo I J, Posa A, Behnisch B, Horak G, Zlabinger K, Gyongyosi M. Human recombinant activated protein C-coated stent for the prevention of restenosis in porcine coronary arteries. J Mater Sci Mater Med 2015; 26(10):241.
13. Foo R S, Gershlick A H, Hogrefe K, Baron J H, Johnston T W, Hussey A J, Garner I, de Bono D P. Inhibition of platelet thrombosis using an activated protein C-loaded stent: in vitro and in vivo results. Thromb Haemost 2000; 83(3):496-502.

What is claimed is:

1. A vascular stent, wherein the vascular stent is loaded with an endothelial protein C receptor (EPCR) activator and a limus drug, and the manner of loading comprises but not limited to spraying and wrapping.

2. The vascular stent according to claim 1, wherein the EPCR activator is one or more of activated protein C, TR47 and Parmodulin 2.

3. The vascular stent according to claim 1, wherein the limus drug comprises one or more of rapamycin, everolimus, zotarolimus, tacrolimus, deforolimus, biolimus A9 and pimecrolimus.

4. The vascular stent according to claim 1, wherein the spraying comprises but not limited to spraying a coating of an EPCR activator and a limus drug on a metal stent; and the wrapping comprises but not limited to wrapping the EPCR activator and the limus drug in a bioresorbable stent.

5. The vascular stent according to claim 1, wherein the spraying amounts of the EPCR activator and the limus drug are 1 μg/mm-100 μg/mm and 1 μg/mm-100 μg/mm respectively.

6. The vascular stent according to claim 1, wherein the wrapping amounts of the EPCR activator and the limus drug are 1 μg/mm-100 μg/mm and 1 μg/mm-100 μg/mm respectively.

7. A manufacturing process for the vascular stent according to claim 1, comprising the following steps:
S1, performing stent pretreatment:
S2, preparing an EPCR activator-limus drug solution: dissolving the limus drug and the EPCR activator in a tetrahydrofuran solution containing 10% of poly(lactic-co-glycolic acid) (PLGA); and
S3, performing rotary spraying.

8. The manufacturing process according to claim 7, wherein final concentrations of the limus drug and the EPCR activator in S2 are both 50 μg/mL.
